(19) United States
(12) Patent Application Publication
(10) Pub. No.: US 2020/0219468 A1
(43) Pub. Date: Jul. 9, 2020

(54) HEAD MOUNTED DISPLAYING SYSTEM AND IMAGE GENERATING METHOD THEREOF

(71) Applicant: HTC Corporation, Taoyuan City (TW)

(72) Inventors: Chien-Min Wu, Taoyuan City (TW); Huan-Hsin Li, Taoyuan City (TW); Cheng-Han Hsieh, Taoyuan City (TW)

(73) Assignee: HTC Corporation, Taoyuan City (TW)

(21) Appl. No.: 16/702,548

(22) Filed: Dec. 4, 2019

Related U.S. Application Data
(60) Provisional application No. 62/774,880, filed on Dec. 4, 2018.

(51) Int. Cl. G09G 5/37; G06F 3/01; G09G 5/10; G09G 5/02; G16H 50/20; A61B 5/00; A61B 5/024; A61B 5/11

(52) U.S. Cl. CPC G09G 5/37; G09G 2354/00; G09G 5/10; G09G 5/02; G16H 50/20; A61B 5/6803; A61B 5/7445; A61B 5/7455; A61B 5/7267; A61B 5/02438; A61B 5/02405; A61B 5/4866; A61B 5/4884; A61B 5/11; G06F 3/011

(57) ABSTRACT

A head mounted displaying system and an image generating method thereof are provided. The head mounted displaying system includes a displaying device, a movement sensor, a frame, an image generating system and a physiological information sensor. The movement sensor senses a movement of an object or senses a movement of the displaying device. The frame is configured to fix the displaying device. The image generating system is coupled to the displaying device. The image generating system displays an image through the displaying device. The image includes a first part. The first part is irrelative to a sensing result of the movement sensor. The physiological information sensor is disposed at the frame and coupled to the image generating system. The image generating system adjusts the first part of the image displayed by the displaying device according to physiological information sensed by the physiological information sensor.

### CROSS-REFERENCE TO RELATED APPLICATION

This application claims the priority benefit of U.S. provisional application Ser. No. 62/774,880, filed on Dec. 4, 2018. The entirety of the above-mentioned patent application is hereby incorporated by reference herein and made a part of this specification.

### TECHNICAL FIELD

The invention relates to a system and a method, and more particularly, to a head mounted displaying system and an image generating method thereof.

### BACKGROUND

In recent years, head mounted display (HMD) devices, such as Augmented Reality (AR), Mixed Reality (MR), or Virtual Reality (VR) displays, have gradually become a popular product on the market. With the development of science and technology, virtual reality technology has been increasingly used in life. In the prior art, when a user wishes to obtain a realistic interaction experience with a real object in a virtual environment, it is often necessary to adjust an image display mode or an interaction mode through a specially set method. When the user needs to interact with the real object in a virtual reality environment, such as playing a game, the existing technology cannot provide a convenient interactive method for playing the game in a virtual world. Therefore, there is a need for a method that can accurately analogize the interaction between the user and the real object. The information disclosed in this Background section is only for enhancement of understanding of the background of the described technology and therefore it may contain information that does not form the prior art that is already known to a person of ordinary skill in the art.

### SUMMARY

The invention provides a head mounted displaying system and an image generating method thereof capable of solving the problem regarding lack of correlation between images and physiological information of users.

The head mounted displaying system of the invention includes a displaying device, a movement sensor, a frame, an image generating system and a physiological information sensor. The movement sensor senses a movement of an object or senses a movement of the displaying device. The frame is configured to fix the displaying device. The image generating system is coupled to the displaying device. The image generating system displays an image through the displaying device. The image includes a first part. The first part is irrelative to a sensing result of the movement sensor. The physiological information sensor is disposed at the frame and coupled to the image generating system. The image generating system adjusts the first part of the image displayed by the displaying device according to physiological information sensed by the physiological information sensor.

In the image generating method of the head mounted displaying system of the invention, the head mounted displaying system includes a displaying device and a movement sensor. The movement sensor senses a movement of an object or senses a movement of the displaying device. The image generating method of the head mounted displaying system includes: sensing the movement of the object or sensing the movement of the displaying device; sensing physiological information; and adjusting a first part of an image displayed by the displaying device according to the physiological information, the first part being irrelative to a sensing result of the movement sensor.

Based on the above, in the head mounted displaying system and the image generating method thereof in the invention, the generated image may be adjusted according to the physiological information, and respond may be made according to a physiological condition of the user in real time.

### BRIEF DESCRIPTION OF THE DRAWINGS

FIG. 1 is a schematic diagram of a head mounted displaying system in an embodiment of the invention.

FIG. 2 is a schematic diagram of inner surface of the frame in FIG. 1.

FIG. 3 is a schematic diagram for explaining a part related to an image displayed by the displaying device and a sensing result of the movement sensor.

FIG. 4 is a diagram illustrating a correspondence relationship between the physiological information sensor of FIG. 1 and a user's forehead.

FIG. 5 is a schematic diagram of a head mounted displaying system in another embodiment of the invention.

FIG. 6 is a flowchart of an image generating method in an embodiment of the invention.

FIG. 7 is a flowchart of an image generating method in another embodiment of the invention.

### DESCRIPTION OF THE EMBODIMENTS

The exemplary embodiments will now be described more fully with reference to the accompanying drawings. However, the exemplary embodiments may be implemented in various forms and should not be construed as being limited to the examples set forth herein; rather, these embodiments are provided so that this invention will be more comprehensive and complete, and will fully convey the concept of the exemplary embodiments to those skilled in the art. The described features, structures, or characteristics may be combined in any suitable manner in one or more embodiments. In the following description, numerous specific details are set forth in order to provide a thorough understanding of the embodiments of the invention. However, those skilled in the art will realize that the technical solutions of the invention may be practiced by with one or more of the specific details omitted, or with other methods, compositions, devices, steps and the like adopted. In other cases, the conventional technical solutions are not shown or described in detail to avoid overwhelming and obscure aspects of the invention.

In addition, the drawings are merely schematic illustrations of the invention and are not necessarily drawn to scale. The same symbols in the drawings represent the same or similar parts, and thus repeated descriptions thereof will be omitted. Certain blocks shown in the drawings are functional entities and do not necessarily have to correspond to physically or logically independent entities. These functional entities may be implemented in software, or implemented in one or more hardware modules or integrated circuits, or these functional entities may be implemented in different networks and/or processor devices and/or microcontroller devices.

The head mounted displaying system includes a head mounted displaying device and an image displaying system. The head mounted displaying device includes a frame, and the frame may include a displaying device and a pair of extending portions. One end of the extending portion may be connected to the displaying device, and configured to fix the displaying device to a visible range of a user. The displaying device may cover the eyes of the user, and may include an optical system (not illustrated) and a protective casing. The displaying device may be a built-in displaying device or an external portable displaying device (e.g., a smart phone or the like). The displaying device may be a closed display system or an open glasses. The head mounted displaying device may be independent of the image displaying system or integrated with the image displaying system into one device. For example, the image displaying system may be integrated with the head mounted displaying device as the head-mounted displaying system in the smart phone. The image displaying system may be a computer system, a cloud device or an edge computing device, which is structurally separated from the head mounted displaying device and accesses data using a wireless connection. The type of the displaying device may be adjusted according to the application of a head mounted displaying system 100 in a virtual reality system, an augmented reality system, or a mixed reality system. The optical system includes an optical element for changing a light path of the displaying device, such as a lens, a light guide or a prism. The invention is not limited in this regard.

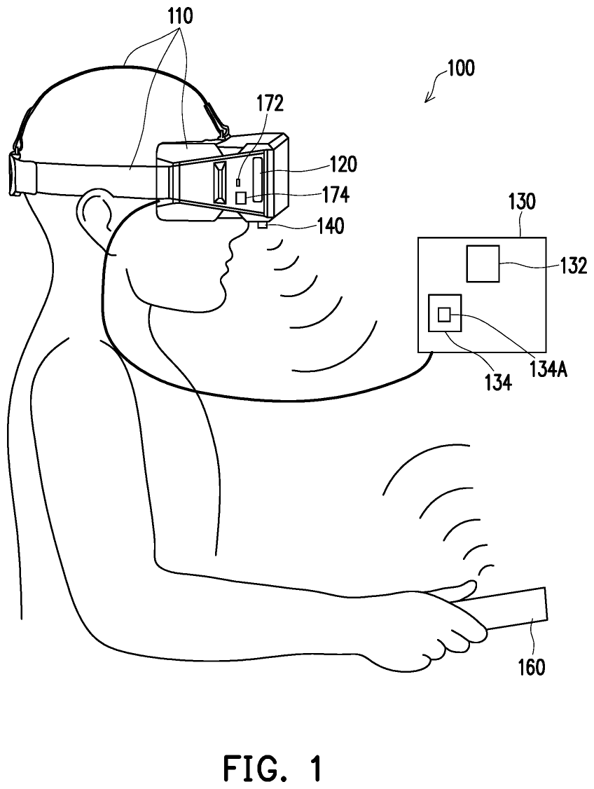

FIG. 1 is a schematic diagram of a head mounted displaying system in an embodiment of the invention. Referring to FIG. 1, the head mounted displaying system 100 of the present embodiment includes a displaying device 120, a movement sensor 140, a frame 110, an image generating system 130 and a physiological information sensor 150. The movement sensor 140 of the present embodiment senses a movement of an object. However, the movement sensor in other embodiment may sense a movement of the displaying device. The frame 110 is used to fix the displaying device 120 and may be fixed onto a user's head during use. The image generating system 130 is coupled to the displaying device 120. The image generating system 130 displays an image through the displaying device 120. The image includes a first part. The first part is irrelative to a sensing result of the movement sensor 140. The image mentioned here may be applied to augmented reality, mixed reality, virtual reality or other forms of reality.

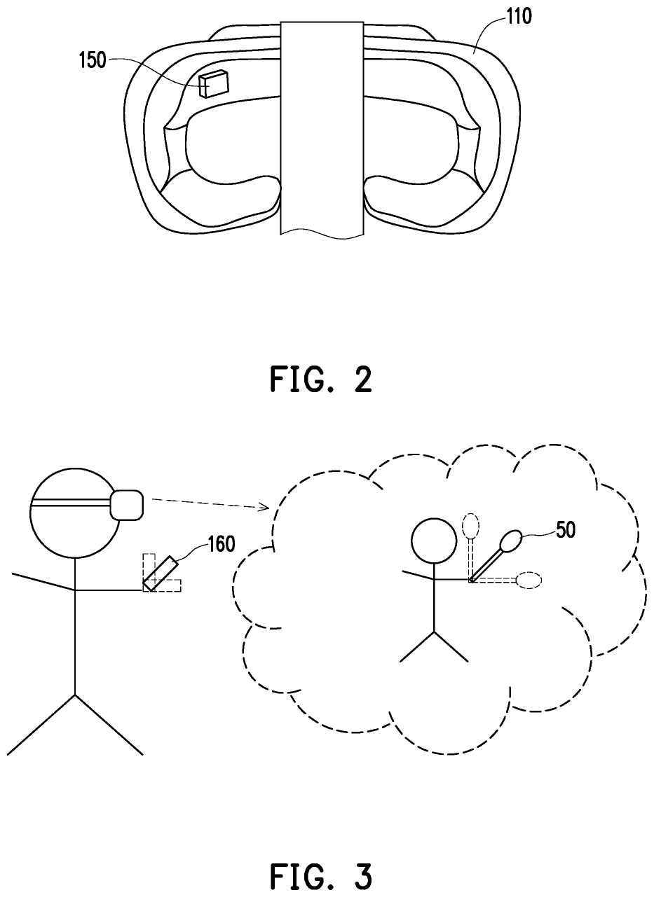

FIG. 2 is a schematic diagram of inner surface of the frame in FIG. 1. Referring to FIG. 1 and FIG. 2, the physiological information sensor 150 of the present embodiment is disposed at the frame 110 and coupled to the image generating system 130. The image generating system 130 adjusts the first part of the image displayed by the displaying device 120 according to physiological information sensed by the physiological information sensor 150.

The displaying device 120 may be a screen, a projection device, an LCD, a light field displaying device, or other displaying devices.

The movement sensor 140 of the present embodiment may be disposed at the frame 110. In other embodiments, the movement sensor may be disposed in a controller, or disposed in the displaying device 120. The movement sensor 140 of the present embodiment may be used to detect the movement of a user's hand, foot, or torso. In other embodiments, the movement sensor may also be independent of the frame to capture a movement of the user by using use a camera, and may be provided with a wireless device to transmit data. The movement sensor 140 may be a camera, and may also be a light, electrical, magnetic, gravity, acceleration, or ultrasonic sensor.

The physiological information sensor 150 may also be an independent accessory that can be connected to the frame 110 through an electrical connection port (such as a TYPE C port or a USB port).

Because the physiological information sensor 150 can provide the physiological information of the user to the image generating system 130, the image generating system 130 is able to adjust the image displayed by the displaying device 120 according to status and changes of the physiological information, provide more immersive experiences, actively adjust the image to achieve better exercise effects, viewing experience and other purposes for users, and reduce possibility of the image causing discomfort to the user.

The image generating system 130 may include a processing unit 132. The processing unit processes and outputs display data. The display data is displayed as the image by the displaying device 120. The display data includes first data and second data. The first data is displayed as the first part of the image by the displaying device 120. The second data is displayed as a second part of the image by the displaying device 120.

FIG. 3 is a schematic diagram for explaining a part related to an image displayed by the displaying device and a sensing result of the movement sensor. Referring to FIG. 1 and FIG. 3, the image generating system 130 described above adjusts the first part of the image displayed by the displaying device 120 according to the physiological information sensed by the physiological information sensor 150, and the first part is irrelative to the sensing result of the movement sensor 140. Herein, the so-called “irrelative” refers to that the image or an audio-visual feature will not be affected by the sensing result of the movement sensor. In addition, the image displayed by the displaying device 120 further includes the second part, and the second part changes in response to a movement of the movement sensor 140. Specifically, it is assumed that the user makes a swing of a controller 160 in hand when the user is playing a tennis game. As can be seen from the image viewed by the user from the displaying device 120 shown on the right side in FIG. 3, a tennis racket 50 will swing with the swing of the controller 160, and the swing of the controller 160 is sensed and obtained by the movement sensor 140. Therefore, the swing or the movement of the tennis racket 50 in the image displayed by the displaying device 120 belongs to the second part related to the sensing result of the motion sensor 140 and does not belong to the first part in the image. However, a speed and a trajectory of a ball returned by a game opponent in the image (or the resolution or the display frequency of the overall image) will not be different due to the movement of the controller 160 sensed by the movement sensor 140, and they belong to the first part of the image. Overall, the image generating system 130 may generate a plurality of events and generate the image displayed by the displaying device 120 according to these events. These events are, for example, targets that appear in a shooting game. The image generating system 130 adjusts a generation frequency of the events according to the physiological information sensed by the physiological information sensor 150. Therefore, when a heart rate of the user is determined to be too high from the physiological information, an occurrence frequency of the targets in the shooting game may be reduced. On the contrary, the occurrence frequency of the targets in the shooting game may be increased to help maintaining the heart rate of the user within a proper range.

Similarly, image or video features (an update frequency, a contrast, a brightness, a color difference, a white balance, a tone curve, a color balance, a color saturation, a color temperature, a color difference correction, an image size, resolution, a volume, a sound frequency range, a loudness, a pitch, a sound quality, a frequency, an amplitude, harmonics, or the like in the image or the video) will not be different due to the sensing result of the movement sensor 140, and these features belong to the first part of the image. The image generating system 130 adjusts the feature of the first part of the image according to the physiological information sensed by the physiological information sensor 150. In addition, the image generating system 130 may also adjust the content to a plot development route in the image of the interactive movie.

The head mounted displaying system 100 of the present embodiment may further include at least one of a speaker 172 and a vibrator 174. Accordingly, the image generating system 130 may adjust the at least one of a sound and a vibration generated by the image generating system 130 according to the physiological information of the user provided by the physiological information sensor 150. For example, in addition to adjusting the image, the image generating system 130 may also change a mode and an intensity of the vibration or music corresponding to the image to increase or decrease a nervous feeling of the user. Alternatively, the volume, the sound frequency range, the loudness, the pitch, the sound quality, the frequency, the amplitude and the harmonics of the sound made by the speaker corresponding to the first part of the image (the first part is irrelative to the sensing result of the movement sensor) may also be adjusted. Naturally, data display of the physiological information may also be added simply to the image, so that the user can learn of the physiological condition in real time. More details will be illustrated later.

In this embodiment, the image generating system 130 actively adjusts the image generated by the image generating system 130 according to the physiological information of the user, rather than adjusting the image by accepting instructions of the user. In this embodiment, the image generating system 130 may include an artificial intelligence module 134, and the artificial intelligence module 134 adjusts the image displayed by the displaying device 120 according to the physiological information sensed by the physiological information sensor 150. The artificial intelligence module 134 includes a learning module 134A, so that the image generating module 130 can improve user experiences through a self learning. The learning module 134A generates a function module according to the physiological information sensed by the physiological information sensor 150 and the image displayed by the displaying device 120. The artificial intelligence module 134 adjusts the image displayed by the displaying device 120 according to the physiological information sensed by the physiological information sensor 150 and the function module. The learning module 134A may also generate a function module according to the physiological information sensed by the physiological information sensor 150 and a feature of the image displayed by the displaying device 120.

After receiving the physiological information of the user such as heartbeat, ECG, body temperature, blood oxygen content, blink frequency, the artificial intelligence module 134 may adjust a display refresh rate from 120 Hz to 60 Hz or from 60 Hz to 120 Hz according to the function module generated in advance. In addition, the artificial intelligence module 134 may be trained by using a combination of Long Short-Term Memory (LSTM), Convolutional Neural Network (CNN), Recurrent Neural Network (RNN), Deep Neural Network (DNN) and Capsule Network. The artificial intelligence module 134 may also be trained by supervised or unsupervised methods.

In this embodiment, the physiological information sensor 150 includes at least one of a photoplethysmography (PPG) sensor, an electrocardiography (ECG) sensor, a camera and a skin impedance sensor. The PPG sensor can measure the heart rate of the user, and the image generating system 130 may determine an exercise intensity, a mood, a calorie consumption and the like for the user based on the heart rate. The ECG sensor may measure an electrocardiogram of the user, obtain the heart rate of the user more accurately, and even determine whether the user has abnormal conditions such as arrhythmia. For example, the camera can track an eye ball movement of the user, and adjust the image that the user is gazing at. The skin impedance sensor may learn, for example, an amount of sweat of the user, so as to estimate an amount of exercise of the user or whether the user is in excited or nervous emotions. The types of the physiological information sensor 150 above are merely examples, and the invention is not limited thereto.

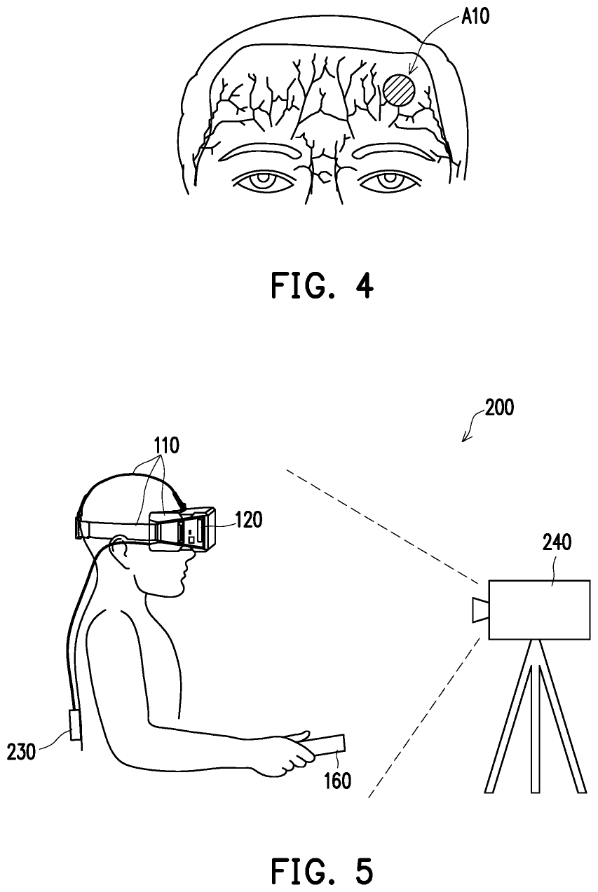

FIG. 4 is a diagram illustrating a correspondence relationship between the physiological information sensor of FIG. 1 and a user's forehead. Referring to FIG. 2 and FIG. 4, the physiological information sensor 150 of the present embodiment may be installed in a position shown by FIG. 2. After the user wears the head mounted displaying system 100, the physiological information sensor 150 may be in contact with the user's forehead, such as an area A10 in FIG. 4. There are many blood vessels distributed on the forehead of a person, and a general user will fix the head mounted displaying system 100 stably on the head when using it. Therefore, the physiological information sensor 150 is able to stably sense the physiological information provided by the blood vessels distributed on the forehead of the user. Compared with the common wrist-worn physiological information sensor that easily loses the sensing ability when the user performs an intense exercise, the physiological information sensor 150 of the present embodiment can provide the sensing result more stably. Naturally, the invention is not intended to limit a installation position of the physiological information sensor 150, and the installation position may be adjusted according to different sensing areas.

FIG. 5 is a schematic diagram of a head mounted displaying system in another embodiment of the invention. Referring to FIG. 5, a head mounted displaying system 200 of the present embodiment is substantially similar to the head mounted displaying system 100 of FIG. 1 and differs in that an image generating system 230 is portable (e.g., able to be worn on the user) and coupled to the displaying device 120 in a wired or wireless manner. In addition, a movement sensor 240 of the present embodiment is separated form the frame 110 and configured to sense a movement of the displaying device 120 as well as a movement of the controller 160. The other parts of the head mounted displaying system 200 of the present embodiment are similar to those of the head mounted displaying system 100 of FIG. 1, and are thus not repeated herein.

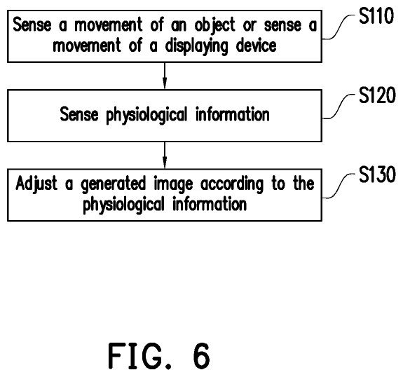

FIG. 6 is a flowchart of an image generating method in an embodiment of the invention. Referring to FIG. 1 and FIG. 4, the image generating method of the present embodiment is applicable to the head mounted displaying system 100 of FIG. 1, the head mounted displaying system 200 of FIG. 5 or other head mounted displaying systems. According to the image generating method of the present embodiment, the movement sensor 140 senses a movement of an object or senses a movement of the displaying device 120 (step S110). Then, the physiological information of a user is sensed (step S120). Next, a first part of an image displayed by the displaying device 120 is adjusted according to the physiological information, and the first part is irrelative to a sensing result of the movement sensor 140 (step S130). For instance, adjustments for the image in step S130 may be performed by the image generating system 130 in FIG. 1. The image generating system 130 is, for example, a computer coupled to the displaying device 120 in a wired or wireless manner. In another embodiment, the image generating method may also be executed by using the head mounted displaying system 100 of FIG. 1, and the image generating system 130 is an external video content providing system such as an online game or a streaming video server that is also coupled to the displaying device 120 in a wired or wireless manner. Then, a video content, such as a content compactness in the online game or a plot development of a streaming video, is adjusted by an external video content provider according to the received physiological information. In step S130, in addition to adjusting the image, at least one of the sound and the vibration may also be adjusted.

Accordingly, the image generating method of the present embodiment can provide users with more immersive experiences, actively adjust the image to help achieving better exercise effect, viewing experience and other purposes for users, and reduce possibility of the image causing discomfort to users.

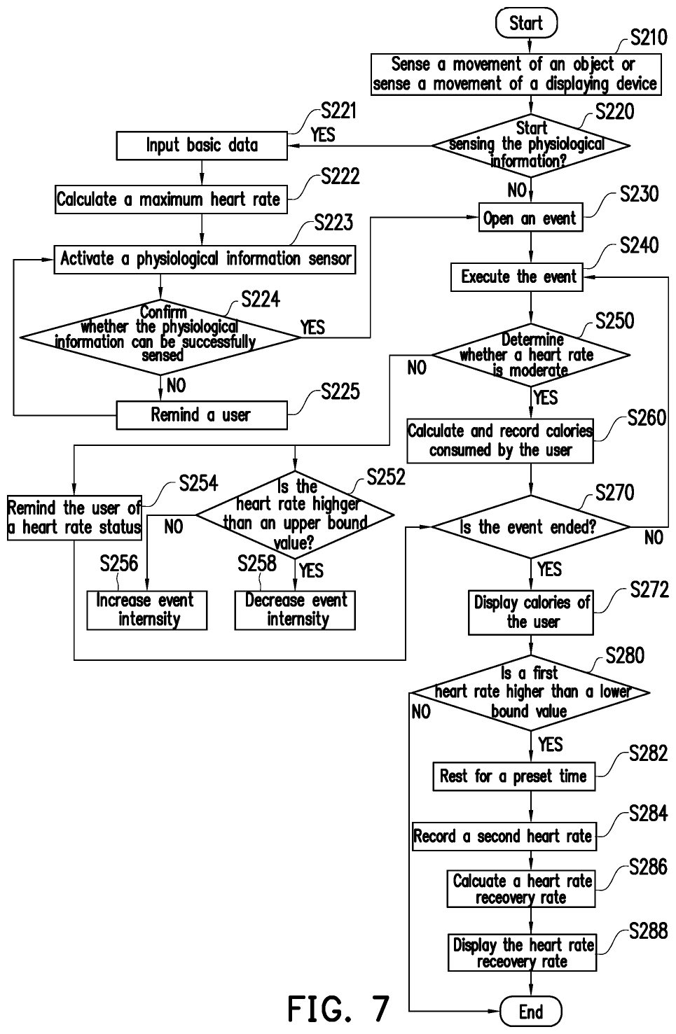

FIG. 7 is a flowchart of an image generating method in another embodiment of the invention. Referring to FIG. 7, here, a tennis game is mainly taken as an example of the event being executed, but the invention is not limited thereto. The image generating method of the present embodiment is also applicable to the head mounted displaying system 100 of FIG. 1, the head mounted displaying system 200 of FIG. 5 or other head mounted displaying systems. According to the image generating method of the present embodiment, in step S210, the movement sensor 140 senses a movement of an object or senses a movement of the displaying device 120. Then, step S220 is performed to ask a user whether to start sensing the physiological information. When the user chooses not to sense the physiological information, the process proceeds to step S230 to start an event, such as starting the tennis game. It should be noted that when the user chooses not to sense the physiological information, after step S230, the process proceeds to step S240 to execute the event without continuing to sense and determine the physiological information.

When the user chooses to sense the physiological information, the process proceeds to step S221 to request the user to input base data. In this embodiment, the basic data includes, for example, at least one of age, height, weight, and gender. Next, in step S222, a maximum heart rate (MHR) is calculated based on the basic data input by the user. The maximum heart rate is obtained by subtracting age of the user from 220, for example.

Next, in step S223, the physiological information sensor is activated. Then, in step S224, whether the physiological information of the user can be successfully sensed is confirmed by, for example, sensing PPG of the user. When confirming that the physiological information of the user cannot be successfully sensed, the process proceeds to step S225 to remind the user to adjust the physiological information sensor so the physiological information of the user can be successfully sensed. For instance, the user may be reminded to confirm whether the head mounted displaying system is being worn firmly, so that the physiological information sensor can successfully sense the physiological information. When confirming that the physiological information of the user can be successfully sensed, the process proceeds to step S230 to start the event, such as starting the tennis game.

Next, the process proceeds to step S240 to execute the event, such as playing the tennis game. During the event, step S250 is performed to determine whether a heart rate in the physiological information is moderate. When the heart rate is not moderate, the process proceeds to step S254, where the generated image is adjusted to remind the user of a heart rate status by, for example, displaying the heart rate of the user in the image. The display method of the heart rate may include directly displaying a value of the heart rate, displaying a degree of the heart rate deviated from a moderate value by a graphic display, or reminding the user, through by text, sound, vibration, image or other forms, about how to adjust the heart rate. The invention is not limited in this regard. Naturally, regardless of whether the heart rate is moderate, a currently accumulated calorie consumption, a current heart rate, a minimum heart rate, the maximum heart rate, an average heart rate, or other physiological information may all be displayed in the image in real time. When the heart rate is not moderate, step S252 may also be performed to further determine whether the heart rate is higher than an upper bound value. If so, the process proceeds to step S258, where the generated image is adjusted to decrease an event intensity so that the heart rate of the user is decreased by, for example, decreasing an intensity of the tennis game. If not, it means that the heart rate is lower than a lower bound value and the process proceeds to step S256, where the generated image is adjusted to increase the event intensity so that the heart rate of the user is increased by, for example, increasing the intensity of the tennis game. For example, a speed at which a game opponent hits back the ball may be increased, or a frequency at which the game opponent hits back the ball may be increased. Here, the upper bound value is, for example, 80% of the maximum heart rate of the user, and the lower bound value is, for example, 60% of the maximum heart rate of the user. When the heart rate is between the upper bound value and the lower bound value, it usually means that the heart rate of the user is within a best exercise fat burning heart rate range, which can effectively help the user to achieve fitness and weight loss in the game. Regarding the way of adjusting the image, with the game as an example, a complexity of the game or a rhythm of the game may be adjusted. Naturally, users can freely choose whether to enable this function. If this function is not activated, the game proceeds with the existing rhythm. Conversely, if this function is activated, the system will dynamically adjust various game-related parameters to increase or decrease the amount of exercise for the user, and then control the heart rate of the user to fall within the best exercise fat burning heart rate range. Taking the tennis game as an example, a ball speed of a tennis ball may be adjusted so that the user can increase or decrease a speed of hitting back the ball. Alternatively, according to position information provided by the head mounted displaying system, a current position of the user can be known, and then a direction of the tennis ball may be controlled so as to increase or decrease steps that the user actually needs to take. Alternatively, according to information provided by a hand held controller cooperating with the head-mounted display system, the user may have to increase or decrease an intensity of the swing so that the tennis ball may be hit further or closer. In other games, other than speeding up or slowing down the pace of the game, the number of enemies that appear at the same time may also be increased or decreased. Or, in a boxing game, methods that can allow the user to increase or decrease the heart rate may include increasing dodge actions, or adjusting parameters so that the user needs to punch faster to increase a chance of hitting and a boxing intensity when hitting.

Further, in a shooting game, when the physiological information indicates that a head temperature of the user reaches a preset highest temperature, the image generating system may decrease a frequency at which the targets appear or a speed at which the target attacks the user according to such temperature variation. In this way, the nervous feeling or use of the head may be effectively reduced for the user, thereby preventing the user from discomfort. The appearance of each target can be regarded as one generated event and the physiological condition of the user may be affected by adjusting the generation frequency of the events.

In addition, if it is determined that the heart rate in the physiological information is moderate in step S250, the process may proceed to step S260 to calculate and record calories consumed by the user. Next, in step S270, whether the event is ended is confirmed. If not, the process returns to step S240 to continue executing the event and monitoring the physiological information of the user. If the event is ended, the process proceeds to step S272 to display the calories consumed by the user in the image.

After the event is ended, step S280 may be performed to determine whether a first heart rate in the physiological information of the user is higher than a lower bound value and record the first heart rate. Here, the lower bound value described is identical to the lower bound value described above, but the invention is not limited thereto. When the first heart rate is not higher than the lower bound value, the measurement ends. When the first heart rate is higher than the lower bound value, the process proceeds to step S282, where a rest starts for a preset time (e.g., 1 to 2 minutes). Then, after the preset time has passed after the first heart rate is recorded, the process proceeds to step S284, where a second heart rate in the physiological information of the user at that time is recorded. Then, the process proceeds to step S286, where a heart rate recovery rate of the user is calculated by using the first heart rate and the second heart rate. Afterwards, in step S288, the heart rate recovery rate of the user is displayed in the image. In addition, when the heart rate recovery rate is unsatisfactory, the head mounted displaying system may also actively send relevant information to a medical or first-aid institution preset by the user, so that the user can get appropriate medical or emergency treatment immediately when in need.

In summary, according to the head mounted displaying system and the image generating method of the invention, not only is the physiological information of the user measured, the generated image is also adjusted based on the measured physiological information. As a result, more immersive experiences may be provided for users, and better exercise effects, viewing experience and other purposes may also be achieved for users.

What is claimed is:

1. A head mounted displaying system, comprising:
a displaying device;
a movement sensor, sensing a movement of an object or sensing a movement of the displaying device;
a frame, fixing the displaying device;
an image generating system, coupled to the displaying device, the image generating system displaying an image through the displaying device, the image comprising a first part, the first part being irrelative to a sensing result of the movement sensor; and
a physiological information sensor, disposed at the frame, the physiological information sensor being coupled to the image generating system, the image generating system adjusting the first part of the image displayed by the displaying device according to physiological information sensed by the physiological information sensor.

2. The head mounted displaying system according to claim 1, wherein the image generating system comprises a processing unit, the processing unit processes and outputs display data, and the display data is displayed as the image by the displaying device.

3. The head mounted displaying system according to claim 2, wherein the display data comprises first data and second data, the first data is displayed as the first part of the image by the displaying device, the second data is displayed as a second part of the image by the displaying device, and the second part of the image changes in response to a movement of the movement sensor.

4. The head mounted displaying system according to claim 1, wherein the image generating system adjusts a feature of the first part of the image according to the physiological information sensed by the physiological information sensor.

5. The head mounted displaying system according to claim 4, wherein the feature is an update frequency, a contrast, a brightness, a color difference, a white balance, a tone curve, a color balance, a color saturation, a color temperature, a color difference correction, an image size or a resolution.

6. The head mounted displaying system according to claim 1, wherein the image generating system generates a plurality of events and generates the image displayed by the displaying device according to the events, and the image generating system adjusts a generation frequency of the events according to the physiological information sensed by the physiological information sensor.

7. The head mounted displaying system according to claim 1, further comprising: a vibrator, the image generating system adjusting a vibration generated by the vibrator according to the physiological information sensed by the physiological information sensor.

8. The head mounted displaying system according to claim 1, wherein the image generating system comprises an artificial intelligence module, and the artificial intelligence module adjusts the image displayed by the displaying device according to the physiological information sensed by the physiological information sensor.

9. The head mounted displaying system according to claim 8, wherein the artificial intelligence module comprises a learning module, the learning module generates a function module according to the physiological information sensed by the physiological information sensor and the image displayed by the displaying device, and the artificial intelligence module adjusts the image displayed by the displaying device according to the physiological information sensed by the physiological information sensor and the function module.

10. The head mounted displaying system according to claim 9, wherein the learning module generates a function module according to the physiological information sensed by the physiological information sensor and a feature of the image displayed by the displaying device.

11. An image generating method of a head mounted displaying system, the head mounted displaying system comprising a displaying device and a movement sensor, the movement sensor sensing a movement of an object or sensing a movement of the displaying device, the image generating method of the head mounted displaying system comprising:
sensing the movement of the object or sensing the movement of the displaying device;
sensing physiological information; and
adjusting a first part of an image displayed by the displaying device according to the physiological information, the first part being irrelative to a sensing result of the movement sensor.

12. The image generating method of the head mounted displaying system according to claim 11, wherein adjusting the first part of the image is to adjust a feature of the first part of the image.

13. The image generating method of the head mounted displaying system according to claim 11, wherein adjusting the first part of the image comprises generating a plurality of events, generating the image displayed by the displaying device according to the events, and adjusting a generation frequency of the events.

14. The image generating method of the head mounted displaying system according to claim 11, wherein adjusting the first part of the image comprises generating a function module according to the sensed physiological information and the image displayed by the displaying device, and adjusting the image displayed by the displaying device by the artificial intelligence module according to the sensed physiological information and the function module.

15. The image generating method of the head mounted displaying system according to claim 11, wherein adjusting the first part of the image comprises generating a function module according to the sensed physiological information and the image displayed by the displaying device, and adjusting the image displayed by the displaying device according to the sensed physiological information and the function module.

16. The image generating method of the head mounted displaying system according to claim 11, sequentially comprising:
sensing the movement of the object or sensing the movement of the displaying device;
asking a user whether to start sensing the physiological information;
requesting the user to input basic data;
calculating a maximum heart rate based on the basic data input by the user;
activating a physiological information sensor and confirming whether the physiological information of the user can be successfully sensed;
when confirming that the physiological information of the user cannot be successfully sensed, reminding the user to adjust the physiological information sensor so the physiological information of the user can be successfully sensed;
when confirming that the physiological information of the user can be successfully sensed, executing an event to generate the image displayed by the displaying device; and
adjusting the first part of the image according to the sensed physiological information.

17. The image generating method of the head mounted displaying system according to claim 11, sequentially comprising:
sensing the movement of the object or sensing the movement of the displaying device;
sensing the physiological information of a user;
executing an event to generate the image displayed by the displaying device;
during the event, determining whether a heart rate in the sensed physiological information is moderate;
when the heart rate in the sensed physiological information is higher than an upper bound value or lower than a lower bound value, adjusting the first part of the image to remind the user of a heart rate status; and
ending the event.

18. The image generating method of the head mounted displaying system according to claim 11, sequentially comprising:
sensing the movement of the object or sensing the movement of the displaying device;
sensing the physiological information of a user;
executing an event to generate the image displayed by the displaying device;
during the event, determining whether a heart rate in the sensed physiological information is moderate;
when the heart rate in the sensed physiological information is higher than an upper bound value, adjusting the first part of the image to decrease the heart rate of the user;
when the heart rate in the sensed physiological information is lower than a lower bound value, adjusting the first part of the image to increase the heart rate of the user; and
ending the event.

19. The image generating method of the head mounted displaying system according to claim 18, wherein the upper bound value is 80% of a maximum heart rate of the user, and the lower bound value is 60% of the maximum heart rate of the user.

20. The image generating method of the head mounted displaying system according to claim 18, further comprising:
when the heart rate in the sensed physiological information is moderate, calculating and recording calories consumed by the user.

21. The image generating method of the head mounted displaying system according to claim 20, wherein after ending the event, the image generating method further comprises:
displaying the calories consumed by the user in the image.

22. The image generating method of the head mounted displaying system according to claim 11, wherein after adjusting the first part of the image according to the sensed physiological information, the image generating method further comprises:
ending an event and determining whether a first heart rate in the physiological information of a user is higher than a lower bound value;
when the first heart rate is higher than the lower bound value, recording the first heart rate;
after the first heart rate is recorded for a preset time, recording a second heart rate in the physiological information of the user, and calculating a heart rate recovery rate of the user by using the first heart rate and the second heart rate; and
displaying the heart rate recovery rate of the user in the image.

23. The image generating method of the head mounted displaying system according to claim 11, wherein in addition to adjusting the first part of the image according to the sensed physiological information, the image generating method further comprises: adjusting at least one of a sound or a vibration.
